(19) United States
(12) Patent Application Publication
(10) Pub. No.: US 2020/0373001 A1
(43) Pub. Date: Nov. 26, 2020

(54) SYSTEM WITH WEARABLE SENSOR FOR DETECTING EEG RESPONSE

(71) Applicant: Thought Beanie Limited, Bristol (GB)

(72) Inventors: Simon HARRISON, Bristol (GB); Alyn MORGAN, Bristol (GB)

(73) Assignee: Thought Beanie Limited, Bristol (GB)

(21) Appl. No.: 16/766,334

(22) PCT Filed: Nov. 23, 2018
(86) PCT No.: PCT/EP2018/082387

§ 371 Date: May 22, 2020

(30) Foreign Application Priority Data
Nov. 24, 2017 (GB) 1719574.4

(51) Int. Cl. G16H 20/70; A61B 5/0478; A61B 5/04; A61B 5/00; A61B 5/0484; A61B 5/16; A61B 5/0482; G16H 40/67; G16H 50/20; G16H 50/70; G06Q 30/06; G06F 1/16; G06F 16/27; G06N 20/00; G06F 3/01

(52) U.S. Cl. CPC G16H 20/70; G06F 2203/011; A61B 5/04004; A61B 5/0006; A61B 5/04842; A61B 5/165; A61B 5/7282; A61B 5/04845; A61B 5/7475; A61B 5/0482; A61B 5/6803; G16H 40/67; G16H 50/20; G16H 50/70; G06Q 30/0631; G06F 1/163; G06F 16/27; G06N 20/00; G06F 3/015; A61B 2503/12; A61B 2562/046; A61B 5/0478

(57) ABSTRACT

A system in which a head-mountable wearable device detects a real-time electroencephalographic (EEG) response from a user while the user is performing an activity or exposed to an external stimulus in a real-world (non-clinical) setting. The wearable device performs on-board processing of a detected EEG signal to enable efficient data wireless transfer to a processing unit (e.g. on a smartphone or the like). The processing unit transforms the EEG signal in real time into a meaningful indicator of current mental state, and presents indicator to the user, e.g. in a form able to improve their performance of the activity, promote complementary activities or to enhance or alter their mental state.

### CROSS REFERENCE TO RELATED APPLICATIONS

This is a U.S. National Phase application under 35 U.S.C. § 371 of International Patent Application No. PCT/EP2018/082387, filed Nov. 23, 2018, which claims priority of United Kingdom Patent Application No. 1719574.4, filed Nov. 24, 2017. The entire contents of which are hereby incorporated by reference.

### FIELD OF THE INVENTION

The invention relates to a system for detecting an electroencephalographic (EEG) response from a user in real time while the user is participating in a real world or virtual activity, e.g. consuming media content, or travelling through a retail environment. In particular, the invention relates to a system in which a detected EEG response of a user exposed to external stimuli can be used to map emotional reactions of the user on to corresponding external stimuli, e.g. to create an emotional or neurofeedback profile for the user. The emotional profile may be used to inform suggestions for future activities or external stimuli to enhance or provide a desired emotional state in the user.

### BACKGROUND TO THE INVENTION

Wearable technology for monitoring physiological properties of a user during an activity is a recent and popular phenomenon. Wearable sensors may be self-contained, or may interface with other accessories, such as smartphones, smartwatches, tablet computers or the like. Collected information may be used to monitor performance and influence training, etc.

US 2015/0297109 discloses a wearable device which detects an electroencephalographic (EEG) response from a user while listening to a musical piece. The EEG response may be used to categorize and tag the musical piece according to the mood it instils in the user.

### SUMMARY OF THE INVENTION

At its most general the present invention provides a system in which a wearable device detects a real-time electroencephalographic (EEG) response from a user while the user is performing an activity or exposed to an external stimulus in a real-world (non-clinical) setting, and capable of transforming the EEG response into a meaningful indicator of current mental state, and presenting that indicator to the user, e.g. in a form able to improve their performance of the activity, promote complementary activities or to enhance or alter their mental state.

The system presented herein may utilize a wearable sensor that can be incorporated (e.g. integrally formed with or mounted within) existing conventional headwear, e.g. sports headwear, such as a cap, a helmet, or their social equivalents etc. The wearable sensor may be configured with a multi-channel sensing unit arranged to wirelessly communicate with a base station processing unit, which may be a smartphone, tablet computer or other portable computing device.

According to the invention, there is provided a system comprising: a wearable sensor comprising: a sensor array for detecting an electroencephalographic (EEG) signal from a user wearing the wearable sensor; a communication unit for wirelessly transmitting the EEG signal; a processing unit arranged to receive the EEG signal transmitted from the head-mountable wearable sensor, the processing unit comprising an analyser module arranged to generate, based on the EEG signal, output data that is indicative of mental state information for the user, wherein the wearable sensor is incorporated into headgear worn by the user exposed to an external stimulus, whereby the output data provides real-time mental state information for the user while exposed to the external stimulus. In use, the invention may thus provide a computing device that is capable of generating, in real-time, output data that is indicative of a user's mental state whilst receiving some stimulus, which may be sight, sound, smell or any combination thereof.

The head-mountable wearable sensor may further comprise a filter module arranged to recognise and remove artefact waveforms from the EEG signal to generate a filtered EEG signal, wherein the communication unit wirelessly transmits the filtered EEG signal.

The filter module may be arranged to apply a recognition algorithm to the EEG signal to filter out waveforms associated with certain artefacts, and wherein the filter module is adapted to update the recognition algorithm using specific waveform for each type of artefact obtained for the user.

The output data may be used in a variety of ways.

In one example it is correlated with the external stimulus in order to create an emotional history profile for the user, which links their mental state with certain stimulus. The correlated information may be stored in a repository where it may be accessible to assist in determining a recommended action or stimulus for the user in future.

In another example, the output data may be used to assist the user in enhancing or altering their mood. This may be done with reference to data in the repository.

In another example, the output data may be used to assist the user in indicating how the external stimulus has affected them, e.g. by way of sharing on social media, applying a rating or score, etc. The user may choose not to be aware of how they are impacted and automatically share their mental state, e.g. in television contests, whether as a judge, member of the audience or watching remotely.

The processing unit may comprise a correlator module arranged to correlate the mental state information with the external stimulus. For example, the processing unit may be arranged to time stamp the mental state information, and synchronise the time stamped mental state information with data indicative of the external stimulus. The data indicative of the external stimulus may comprise a time series of annotatable events that correspond to the external stimulus, or, where the external stimulus is consumption of media content it may comprise a data file indicative of that media content. Where the external stimulus comprises exposure to media content, the correlator module may be arranged to synchronise the mental state information with the media content.

As mentioned above, the system may comprise a repository for storing the correlated mental state information. The repository may be a database or other storage device accessible to the processing unit, e.g. via a network or wireless communication channel.

The system may comprise a portable computing device arranged to execute a user interface application to enable user interaction with the output data. The portable computing device may be any suitable user terminal, e.g. smartphone, tablet computer, laptop computer, etc., that is capable of communication over a data network. The portable computing device may be in wireless communication with the wearable sensor. The processing unit may be part of the portable computing device, whereby the wearable sensor transmits the EEG signal to the portable computing device for subsequent processing. The EEG signal is preferably pre-processed, e.g. filtered by the filter module at the wearable unit, to remove artefacts known to be unrelated to emotion reaction in order to reduce the amount of data that is transmitted.

The user interface application may be arranged to recommend a rating for the external stimulus based on the output data. The user interface application may be arranged to suggest user action based on the output data. The suggested user action comprises any one of: playback and/or streaming of media content, participation in an activity, or selection or purchase of a retail item and/or service, e.g. in a scenario where the repository has a record of retail items and/or services to which the user was previous attracted, based on the mental state information.

The user interface application may be arranged to receive a user input, e.g. an indication of a desired mood, which may be used to determine a suggested user action.

The user interface application may be arranged to compare current output data with historical output data for the user.

Other aspects, options and advantageous features are set out in the detailed description below.

### BRIEF DESCRIPTION OF THE DRAWINGS

Embodiments of the invention are discussed in detail with reference to the accompanying drawings, in which:

FIG. 1 is a schematic view of a system that is an embodiment of the invention;

FIG. 2 is a schematic view of a portable processing unit for mounting in a wearable article for use in an embodiment of the invention;

FIGS. 3A and 3B are front and rear schematic views of a wearable unit that can be used in a first embodiment of the invention;

FIGS. 4A and 4B are front and rear schematic views of a wearable unit that can be used in a second embodiment of the invention;

FIGS. 5A and 5B are front and rear schematic views of a wearable unit that can be used in a third embodiment of the invention;

FIGS. 6A and 6B are front and rear schematic views of a wearable unit that can be used in a fourth embodiment of the invention;

FIGS. 7A and 7B are front and rear schematic views of a wearable unit that can be used in a fifth embodiment of the invention; and

FIG. 8 is a schematic view of a system that is an embodiment of the invention in use.

### DETAILED DESCRIPTION OF THE DRAWINGS

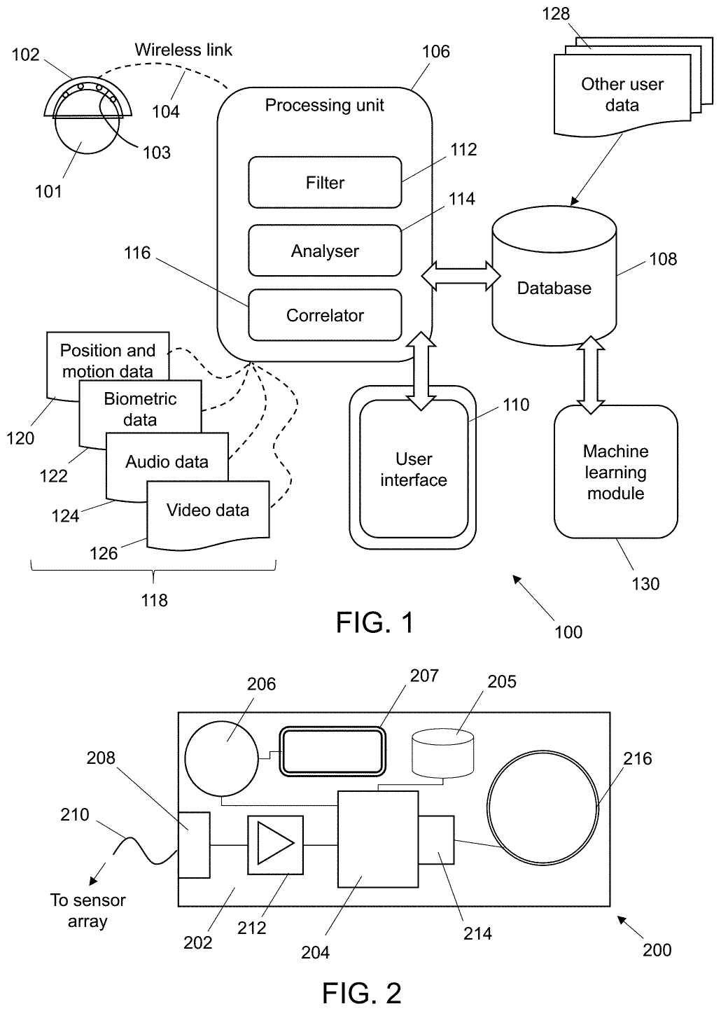

FIG. 1 is a schematic diagram of a system 100 that is an embodiment of the invention. In simple terms, the system 100 comprises three components: (i) a wearable sensor, which may be incorporated into a conventional headgear, e.g. piece of sports equipment (e.g. helmet) or sportswear (e.g. baseball cap) or their social equivalents; (ii) a processing unit, which may be smartphone, smartwatch, tablet or other computing device communicably connected to the wearable sensor; and (iii) a database or other storage or memory facility in communication with the processing unit to provide information that assist analysis of data from the wearable sensor. The three components may be separate from one another or may be located together, in any combination. Similarly, the functions of the processing unit described below may be performed by a plurality of processors in different locations. The processing and/or analysis may thus occur locally, e.g. at a processing unit in the same location as the user, or remotely, e.g. at a processing unit in the cloud or the like.

In FIG. 1, the system 100 comprises a head-mountable wearable device 102 on a user's head 101. The wearable device 102 may be any suitable piece of headwear used when a user performed an activity. A wearable sensor module 103 is mounted or otherwise incorporated or integrated within the headwear. Advantageously, the wearable sensor module of the present invention may be mounted within a standard piece of headgear, which makes the invention readily available for use in real scenarios.

The wearable unit 102 may further comprise one or more audio output elements, e.g. a pair of speakers mounted be at or over a user's ears when wearable sensor module 103 is corrected placed. The speakers may take any suitable form. They may be micro speakers that lie adjacent the user's ears. They may comprise earbuds for locating in the user's ears. They may be in a separate set of headphones worn by the user and wirelessly connected to and/or integrated with the headwear. In another example, the wearable unit 102 may include a display portion, e.g. virtual reality goggles or the like, for mounting over a user's eyes to provide a visual stimulus, e.g. video or still pictures.

The wearable sensor module 103 comprises a sensor array comprising a plurality of sensor elements for obtaining an electroencephalographic (EEG) signal from a user while wearing the headwear. Each sensor element may be arranged to contact the user's scalp to obtain a suitable measurement. The plurality of sensor elements may be located within the headwear at suitable positions for obtaining an EEG signal from suitable nodes across the user's skull. The location of the sensor elements may be selected to facilitate detection of a set of predetermined emotions that are relevant to the activity. For example, the set of predetermined emotion may relate to any one or more emotions that are indicative of emotional valence, i.e. positive and negative emotions such as sadness, happiness, contentment, fear, etc.

The wearable sensor module 103 includes a local processing unit (an example of which is shown in FIG. 2), for controlling the sensor array and generating an EEG signal based on readings from the sensor array. The wearable sensor module 103 may be equipped with a wireless transmitter for transmitting the EEG signal to a remote processing unit 106 for further processing. The wireless transmitter may send the signal over any suitable network using any suitable protocol, e.g. WiFi, Bluetooth®, etc. The wireless transmitter may include 4G or 5G connectivity for immediate transmission and real-time response.

In other examples, the wearable sensor module may include a storage unit, e.g. a computer writable memory such as flash memory or the like, where information can be stored in the headwear and then downloaded and analysed later (e.g. via a wired link). This may be useful where the activity being performed limits or prevents wireless connectivity.

The processing unit 106 is a computing device used to analyse and report on the EEG signal. The processing unit 106 may be arranged to transmit a feedback signal (e.g. a control signal or an audio stream) back to the wearable unit 102 over the wireless link 104. Any computing device capable of receiving the EEG signal from the wearable sensor module may be used. For example, the processing unit 106 may be a smartphone, tablet computer, laptop computer, desktop computer, server computer or the like. The processing unit 106 comprises a memory and a processor for executing software instructions to perform various functions using the EEG signal. In the example illustrated in FIG. 1, the processing unit 106 is shown to have three modules that perform different functions.

The processing unit 106 comprises a filter module 112 arranged to clean up the received EEG signal, e.g. by filtering out environmental artefacts and/or other unwanted frequencies, e.g. associated with unrelated brain activity such as blinking, chewing, moving, irrelevant smelling, etc. The filter module 112 may operate using algorithms arranged to recognise artefact waveforms, e.g. based on input from a normative databases, in the received EEG signal. The algorithms may be adapted to learn the user's specific waveform for each type of artefact, and update the recognition routine accordingly. The filtering process may thus become quicker and more adept with increased use. The wearable unit 102 may comprises a movement sensor (e.g. a pair of accelerometers mounted on either side of the headband). The movement sensor may monitor changes in head position to provide a reference point to assist in removing irrelevant data caused by other types of movement. In one example, the filter module may be arranged to extract data corresponding to target EEG frequency bands from the obtained EEG signal. In this example, the frequency range recorded varies from 1 to 80 Hz, with amplitudes of 10 to 100 microvolts. Recorded frequencies fall into specific groups, with dedicated ranges being more prominent in certain states of mind. The two that are most important for emotional recognition are alpha (8-12 Hz) and beta (12-30 Hz) frequencies. Alpha waves are typical for an alert, but relaxed, state of mind and are most visible over the parietal and occipital lobes. Beta activity evidences an active state of mind, most prominent in the frontal cortex and over other areas during intense focused mental activity.

The processing unit 106 comprises an analyser module 114 that is arranged to process the EEG signal (e.g. after filtering by the filter module 112) to yield information indicative of the user's mental state, e.g. emotional valence. The analyser module 114 may be configured to process the (filtered) EEG signal in a manner such that emotional valence information is effectively generated in real time. To generate the mental state information discussed above, the analyser module 114 may be configured to map the EEG signal onto a mental state vector, whose components are each or are each indicative of an intensity value or probability for a respective emotional state or mental process. The mapping process may be based on a suitable software model drawing on machine learning and artificial intelligence. The analyser module may be arranged to locate unique (but recurring) grades of peak and trough as waves move across the brain. From these recurring signals, the analyser module may identify relevant differentials in hemispheric activation, monitor associated montages, and collate both to clearly evidence emotional valence.

The analyser model may be adaptive to an individual's responses. In other words it may learn to recognise how an individual's detected EEG signals map on to emotional state information. This can be done through the use of targeting sampling and predictive AI techniques. As a result, the analyser module may improve in accuracy and responsiveness with use.

The initial EEG signal obtained using readings from the wearable sensor module 103 may comprise one or more EEG data maps that represent the variation over time of a brainwave electrical signal detected at each sensor location. The EEG data maps may processed to generate responses from each sensor in a plurality of EEG frequency bands (e.g. Alpha, Beta, Theta, etc.). Each sensor may be arranged to capture up to six brainwave frequencies.

In one example, the analyser module 114 may measure asymmetry in the Alpha (confidence) and Beta (composure) EEG bands across the left hemispheric bank to determine positive emotion and make corresponding measurements over the right hemisphere to measure the opposite. An output from this analysis can be indicative of negative anxiety/stress activation in the right prefrontal cortex, amygdala, and insula.

The analyser module 114 is arranged to produce an output data stream in which the emotion-related parameters are identified and time-stamped. The output data stream is delivered to a correlator module 116 effectively as real-time data indicative of a user's current mental status. The mental status information from the analyser module 114 may be transmitted to a repository (e.g. a database 108) where is can be aggregated with other data 128 from the user to form a dataset that can be in turn be used to inform and improve the analysis algorithm, e.g. via a machine learning module 130 that may train a model based on aggregated data in the database 108.

The processing unit 106 may comprise a correlator module 116 that is arranged to correlate or synchronise the EEG signal with other user-related data 118 received at the central processing unit 106. The correlator module 116 may operate to combine the EEG signal with other data before it is processed by the analyser module 114.

The other user-related data 118 may represent an external stimulus or external stimuli experienced by the user while the EEG signal is collected. The external stimuli may be any detectable event that can influence a user's mood. For example, the external stimuli may be related to media content consumed by the user. Media content in this sense may include audio and/or video data, e.g. obtained from streaming and/or download services, DAB radio, e-books, app usage, social media interaction, etc. The other user-related data 118 may thus include information relating to the media content, e.g. audio data 124 and/or video data 126 consumed by the user at the time that the EEG signal was obtained. The audio data may be music or other audio played back e.g. via headphones at the wearable unit 102. Alternatively, the external stimuli may be related to the user's local environment, e.g. including any of sights, sounds and smells that may be experienced. In one example, the user may be in a retail environment (e.g. shopping mall or commercial district), where the external stimuli may be provided by the user's interaction with any of shop fronts, advertising, particular products, purchases, etc. In this example, the other user-related data 118 may include location information, e.g. GPS-based data from a user's smartphone or from suitable detectors (e.g. CCTV cameras or the like) in the retail environment. Images captured by local devices may be analysed to identify a user by applying facial recognition technology or the like. The other user-related data 118 may also include purchase information, such a near field communication (NFC) spending profiles shared by the user from one or more sources, e.g. Apply Pay, PayPal, etc.

The other-user related data 118 may be time-stamped in a manner that enables the correlator module 116 to synchronise it with the EEG signal. This information may be used to annotate the mental state information. Annotation may be done manually or automatically, e.g. by the correlator tagging the audio or video data.

The other user-related data may include biometric data 122 recorded for the user, e.g. from other wearable devices that can interface with the central processing unit 106. The biometric data 122 may be indicative of physiological information, psychological state or behavioural characteristics of the user, e.g. any one or more of breathing patterns, heart rate (e.g. ECG data), blood pressure, skin temperature, galvanic skin response (e.g. sweat alkalinity/conductivity), and salivary cortisol (e.g. obtained from a spit test).

In some examples, the analysis performed by the analyser module 114 may utilise a range of different physiological and mental responses. This may improvement the accuracy or reliability of the output data. For example, the biometric data may be used to sense check the mental state information obtained from the EEG signal.

The other user-related data 118 may include information relating to the external stimulus experienced by the user to assist in matching the user's mental state to specific situations. For example, the other user-related data 118 may include position and/or motion data 120. The position data may be acquired from a global position system (GPS) sensor or other suitable sensors, and may be used to provide information about the location of the user during the activity, e.g. the location within a retail environment. The motion data may be from a motion tracker or sensor, e.g. a wearable sensor, associated with the user. The motion data may be acquired from accelerometer, gyroscopes or the like, and may be indicative or the type and/or magnitude of movement or gesture being performed by the user during the activity. The correlator module 116 of the central processing unit 106 may be able to match or otherwise link the EEG signal with the position data and/or motion data to perform information on physical characteristics of the user whilst exhibiting the observed mental state.

The information obtained as a result of synchronising or tagging the mental state information may be stored in a database 108 to provide a profile for the user, i.e. a personal history or record of measured mental and physiological response during performance of an activity. The analyser module 114 may be arranged to refer to the profile as a means of refining a measurement. In some examples, the analyser module 114 may be arranged to access an aggregated (i.e. multi-user) profile from the database as a means of providing an initial baseline with which to verify or calibrate measurements for a new user.

The processing unit 106 can be accessed by a user interface application 110, which may run on a network-enabled device such as a smartphone, tablet, laptop, etc. The user interface application 110 may be arranged to access information from any of the modules in the processing unit.

For example, the user interface application 110 may be arranged to query information stored in the database 108 in order to present to the user output data. For example, the application 110 may invite the user to indicate a desired mood or emotional state, and then look up from the database 108 one or more external stimuli associated with that mood or emotional state. The identified external stimuli may be presented to the user, e.g. as recommendations to be selected. The recommendations may correspond to consumption of certain media content or a certain retail experience (e.g. purchase).

Additionally or alternatively, the user interface application 110 may be arranged to access emotional state information (e.g. current, or real time, emotional state information) from the analyser module 114. This information may be used to generate output data that can be displayed to the user their current emotional state, or shared by the user, e.g. with their social circle via social media or with other entities for research or commercial purposes, such as retail/lifestyle informatics, or the like. The current emotional state information may also be used to query the database, e.g. to identify one or more external stimuli that could be experienced to enhance, alter or maintain that emotional state. The identified external stimuli may be recommended, e.g. in an automated way, to the user via the user interface application 110.

The system described above may also be arranged to interact with online rating or voting systems, for example to provide a user with an efficient means of registering a score for media content or other external experience. The user interface application 110 may use information from the processing unit to suggest a rating for the user to apply or even to automatically supply a rating based on the relevant emotional state information.

In some examples, the user interface application 110 may offer complementary lifestyle advice and products based on the user's profile.

In the context of media content consumption, the recommendation system discussed above provides a means whereby a user can be exposed to a physical repetition of selected media patterns to achieve a certain emotional response. This can result in imbedded (and quicker) emotional response to the associated media content, as well as improved memory consolidation in respect of the media content.

The functions of the processing unit 106 may be all performed on a single device or may be distributed among a plurality of devices. For example, the filter module 112 may be performed on the wearable unit 102, or a smartphone communicably connected to the wearable unit 102 over a first network. Providing the filter module 112 on the wearable unit, e.g. in advance of amplifying and transmitting the signal may be advantageous in terms of reducing the amount of data that is transmitted and subsequently processed. The analyser module 114 may be provided on a separate server computer (e.g. a cloud-based processor) that is communicably connected to the processing unit 106 over a second network (which may be a wired network). Likewise, the correlator module 116 may be located with the analyser module 114 or separately therefrom.

FIG. 2 is a schematic view of a portable processing unit 200 that can be used in a wearable unit that is an embodiment of the invention. The processing unit 200 comprises a flexible substrate 202 on which components are mounted. The flexible substrate 202 may be mounted, e.g. affixed or otherwise secured, to wearable headgear (e.g. a cap, beanie, helmet, headband or the like).

On the substrate 202 there is a processor 204 that controls operation of the unit, and a battery 206 for powering the unit. The substrate 202 includes an electrode connection port 208 from which a plurality of connector elements 210 extend to connect each sensor element (not shown) to the processing unit 200. The wearable sensor operates to detect voltage fluctuations at the sensor element locations. The processing unit 200 includes an amplification module 212 (e.g. a differential amplifier or the like) for amplifying the voltages seen at the sensors. The amplification module 212 may be shielded to minimise interference.

The processing unit 200 may be configured to take reading from multiple sensors in the array at the same time, e.g. by multiplexing between several channels. In one example, the device may have eight channels, but the invention need not be limited to this number. The voltage fluctuations may be converted to a digital signal by a suitable analog-to-digital converter (ADC) in the processing unit. In one example, a 24-bit ADC is used, although the invention need not be limited to this. The processor 204 may be configured to adjust the number of channels that are used at any given time, e.g. to enable the ADC sampling rate on one or more of the channels to be increased or to switch off channels that have an unusable or invalid output. The ADC sampling rate for eight channels may be 512 Hz, but other frequencies may be used.

The digital signal generated by the processing unit is the EEG signal discussed above. The processing unit 200 includes a transmitter module 214 and antenna 216 for transmitting the EEG signal to the processing unit 106. The transmitter module 214 may be any suitable short to medium range transmitter capable of operating over a local network (e.g. a picocell or microcell). In one example, the transmitter module 214 comprises multi-band (802.11a/b/g/n) and fast spectrum WiFi with Bluetooth® 4.2 connectivity.

The battery 206 may be a lithium ion battery or similar, which can provide a lifetime of up to 24 hours for the device. The battery may be rechargeable, e.g. via a port (not shown) mounted on the substrate 202, or wireless via an induction loop 207.

The processing unit 200 may include a storage device 205 communicably connected to the processor 204. The storage device 205 may be a computer memory, e.g. flash memory or the like, capable of storing the EEG signal or any other data needed by the processing unit 200.

In some examples, the processing unit 200 may be arranged perform the functions of any one or a combination of the filter module 112, analyser module 114 and correlator module 116 discussed above. As mentioned above, it may be particularly advantageous for the filter module 112 to be included in the processing unit 200, e.g. before the amplification module 212, in order to avoid unnecessary processing and transfer of data. The analyser module 114 and correlator module 116 may be provided as part of an app running on a remote user terminal device (e.g. smartphone, tablet, or the like), which in turn may make use of server computers operating in the cloud.

The processing unit 200 may be mounted within the fabric of the headgear within which the wearable sensor is mounted. The electrical connection between the sensor elements and the substrate may be via wires, or, advantageously, may be via a flexible conductive fabric. The conductive fabric may be multi-layered, e.g. by having a conductive layer sandwiched between a pair of shield layers. The shield layers may minimise interference. The shield layers may be waterproof or there may further layers to provide waterproofing for the connections. With this arrangement, the wearable sensor can be mounted in a comfortable manner without sacrificing signal security or integrity.

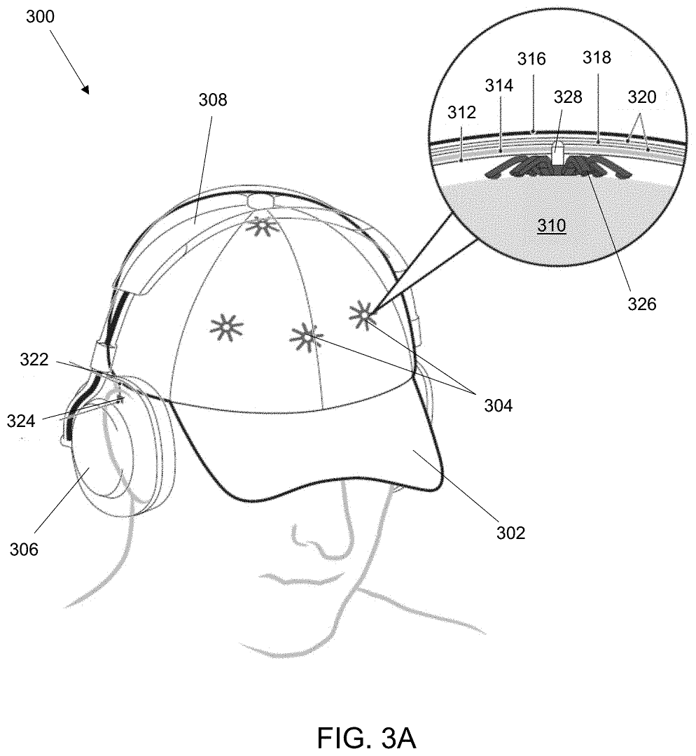

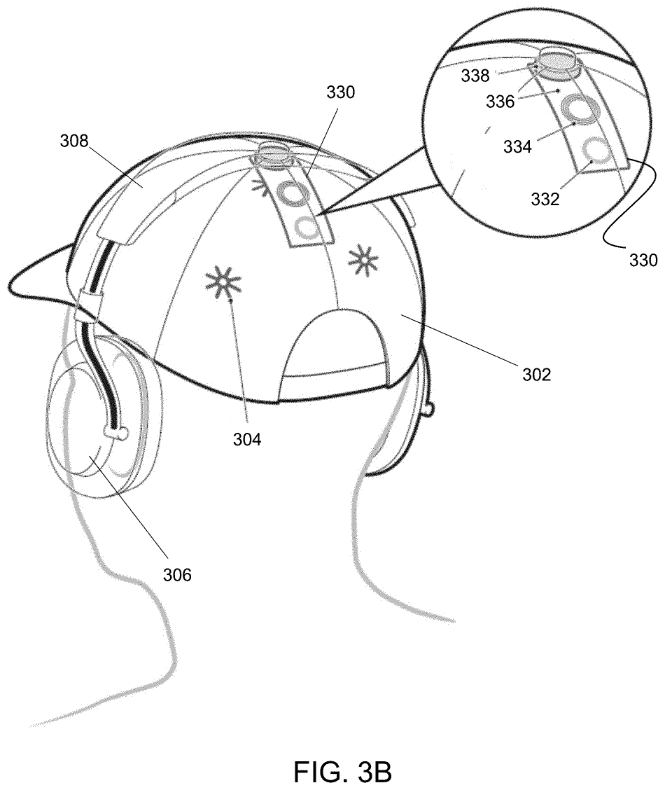

FIGS. 3A and 3B are respectively schematic front and rear diagrams illustrating a wearable unit that can be used in one embodiment of the invention. In this example, the wearable unit comprises a cap 302 and a pair of headphones 306 connected together by a head band 308 that extends over the top of the user's head. As shown in FIG. 3B, in this example a processing unit 330 (which may correspond to the processing unit 200 discussed above) is mounted at the apex of the cap, and curves (or is flexible) to follow the contour of the cap as it extends away from the apex.

A plurality of sensor elements 304 are mounted on an inner surface of the cap 302. The sensor elements 304 are electrically connected to the processing unit 330 by interconnections fabricated within the cap itself.

As shown in a magnified cross-sectional inset of FIG. 3A, this is achieved by forming the cap from a multi-layered structure in which a signal carrying layer 318 is sandwiched between a pair of insulating layers 320, which in turn are between an inner protective layer 312 and an outer protective layer 316. The inner protective layer 312 may be a fabric layer that is in contact with a user's head. On top of the inner protective layer 312 is a layer of foam 314 that protects the user's scalp from unwanted and potentially uncomfortable contact with the conductive layer and processing unit. The signal carrying layer 318 may be formed from a conductive fabric or ink, e.g. a flexible electrically conductive material that electrically connects the sensor elements to the processing unit. The inner and outer insulation layers 320 shield the conductive fabric, e.g. to minimise interference with the signals carried by it. The outer protective layer 316 may be a fabric layer, e.g. formed of any conventional material used for caps.

Each sensor element 304 is mounted on the inner fabric layer 312 such that it contacts the user's head when the cap 302 is worn. Each sensor element 304 comprises a soft deformable body 326 (e.g. formed from dry silicone gel or the like) on which a micro-electrode is mounted to make intimate contact with the user's skin in order to obtain a good signal via the user's skull 310. The micro-electrode extends though the inner fabric layer 312, foam layer 314 and inner insulation layer 320 to contact the conductive fabric layer 318.

A reference electrode 324 is mounted elsewhere on the cap 302 to supply a reference voltage against which the voltage fluctuations are measured. In this example, the reference electrode comprises a graphite pad connected to the processing unit 330 by a fibreglass wire 322.

As shown in a magnified inset of FIG. 3B, the processing unit 330 has a battery 338, wireless charging coil 334 and transmitter 332 mounted on a flexible substrate 336.

The cap 302 and headphones 306 may be separate components, e.g. so that the head band 308 of the headphones can be worn over the cap. Alternatively, the cap 302 and headphones 306 may be part of a single unit.

In use, the processing unit 330 may be in wireless communication with a portable computing device (e.g. smartphone, tablet or the like). The portable computing device may run a user interface application that is arranged to receive information from and transmit information to the processing unit 330. The portable computing device may also be in communication with the headphones, either via the processing unit or via an independent communication channel.

The processing unit 330 may be arranged to transmit an EEG signal to the portable computing device as discussed above, whereupon it may be filtered and analysed to yield mental state information for the user. Information about media content being consumed by the user, e.g. via the headphones 306 can be transmitted or otherwise supplied to the portable computing device.

In some examples, there may be 3 to 7 sensor elements 304 mounted in the cap 302. For example, there may be 2 to 3 dry gel sensors located on the user's frontal lobe when the cap is worn, and 3 to 4 hair-penetrating sensors located on the user's parietal lobe to the rear.

Each sensor element 304 may capture up to 6 brain wave frequencies, thereby monitoring different wave speeds from each. The sensor elements 304 may be spread across various combinations of electrode positions, e.g. F3, F4, FPz, Pz, Cz, P5, P4 in the 10/20 system.

Although not show in FIGS. 3A and 3B, there may be micro-accelerometers on either side of the cap. These may monitor changes in head position associated with the quality of stimuli, and may provide a reference point in removing irrelevant data caused by other types of movement.

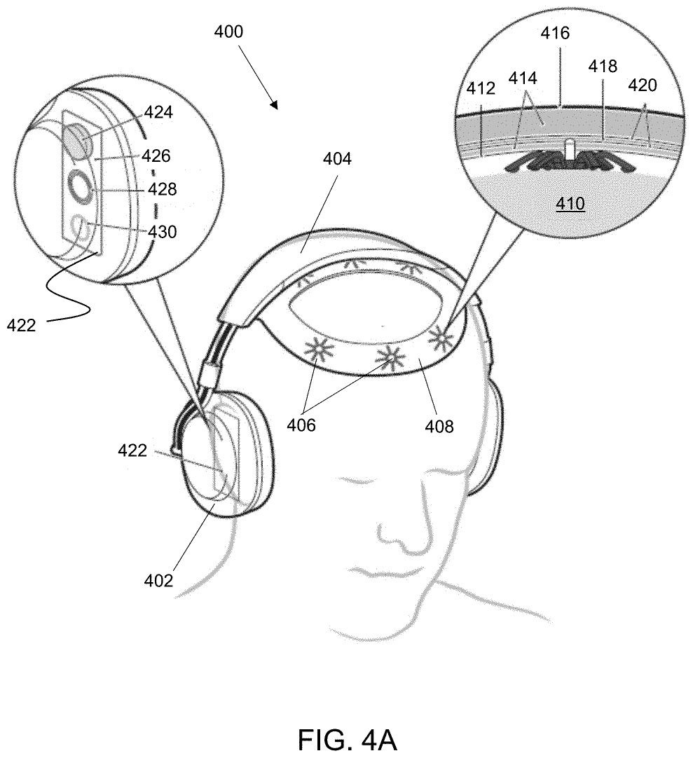

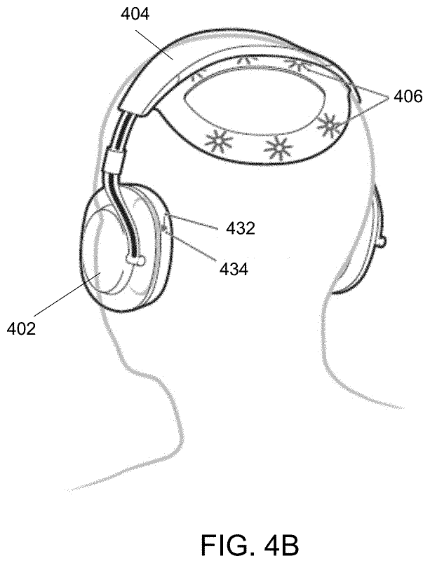

FIGS. 4A and 4B are respectively schematic front and rear diagrams illustrating a wearable unit 400 that can be used in another embodiment of the invention. In this example, the wearable unit comprises headphones 402 with a head band 404 and a halo 408 which sits over a user's head when the headphones 402 are located over their ears. The halo 408 comprises a ring element that has a front loop that passes over the user's frontal lobe, and a rear loop that passes over the user's parietal lobe. The halo 408 may be slidably mounted on an underside of the head band to permit the position of the front loop and rear loop relative to the head band to be adjusted. The halo 408 may be slidable in any one or more of a forward-backward sense, a side-to-side sense, or a rotatable sense.

As shown in FIG. 4A, in this example a processing unit 422 (which may correspond to the processing unit 200 discussed above) is mounted within one of the headphones 402.

A plurality of sensor elements 406 are mounted on an inner surface of the halo 408. The sensor elements 406 are electrically connected to the processing unit 422 by interconnections fabricated within the halo itself, which in turn are connected to signal carriers (e.g. suitable wiring) in or on the head bead and headphones.

As shown in a first magnified cross-sectional inset of FIG. 4A, this is achieved by forming the halo from a multi-layered structure in which a signal carrying layer 418 is sandwiched between a pair of insulating layers 420, which in turn are between an inner protective layer 412 and an outer protective layer 416. The inner protective layer 416 may be a fabric layer that is in contact with a user's head. On top of the inner protective layer 416 is a layer of foam 414 that protects the user's scalp from unwanted and potentially uncomfortable contact with the conductive layer and processing unit. The outer layer 416 may be a rigid shell. A second layer of foam 414 may protect the signal carrying layer 418 from the outer layer 416.

The signal carrying layer 418 may be formed from a conductive fabric or ink, e.g. a flexible electrically conductive material that electrically connects the sensor elements to the processing unit. The inner and outer insulation layers 420 shield the conductive fabric, e.g. to minimise interference with the signals carried by it.

Each sensor element 406 is mounted on the inner fabric layer 412 such that it contacts the user's head when the halo 408 is worn. In a similar manner to that shown in FIG. 3A, each sensor element 406 comprises a soft deformable body on which a micro-electrode is mounted to make intimate contact with the user's skin in order to obtain a good signal via the user's skull 410.

As shown in FIG. 4B, a reference electrode 434 is mounted elsewhere on the unit to supply a reference voltage against which the voltage fluctuations are measured. In this example, the reference electrode comprises a graphite pad connected to the processing unit 422 by a fibreglass wire 432.

As shown in a second magnified inset of FIG. 4B, the processing unit 422 has a battery 424, wireless charging coil 428 and transmitter 430 mounted on a flexible substrate 426.

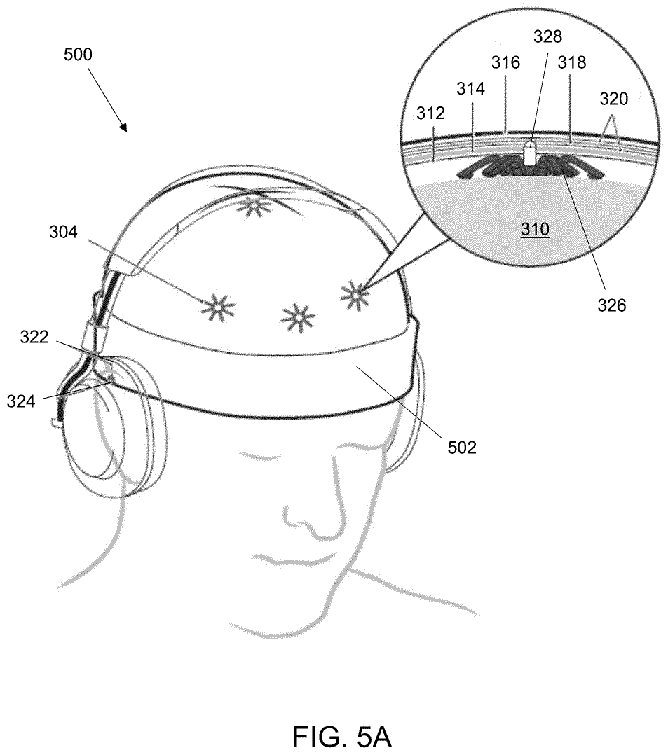

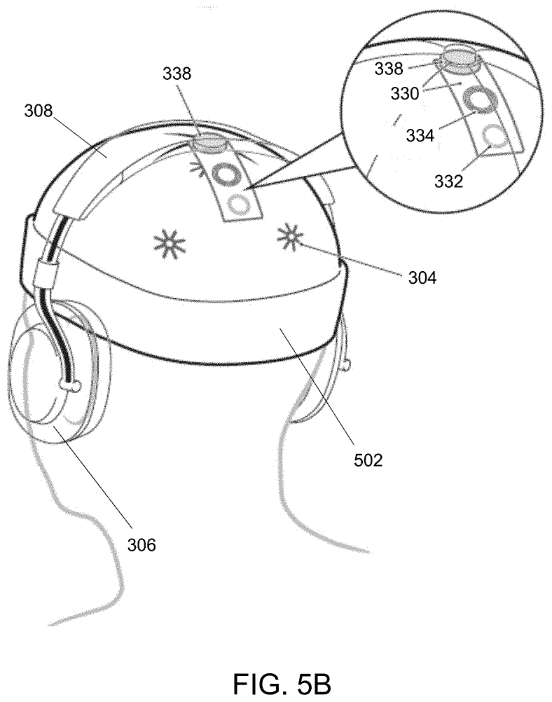

FIGS. 5A and 5B are respectively schematic front and rear diagrams illustrating a wearable unit 500 that can be used in another embodiment of the invention. Features in common with FIGS. 3A and 3B are given the same reference number and are not described again. In this example, the wearable unit 500 comprises a beanie 502 (i.e. a flexible head covering made from elasticated fabric) in place of the cap shown in FIGS. 3A and 3B.

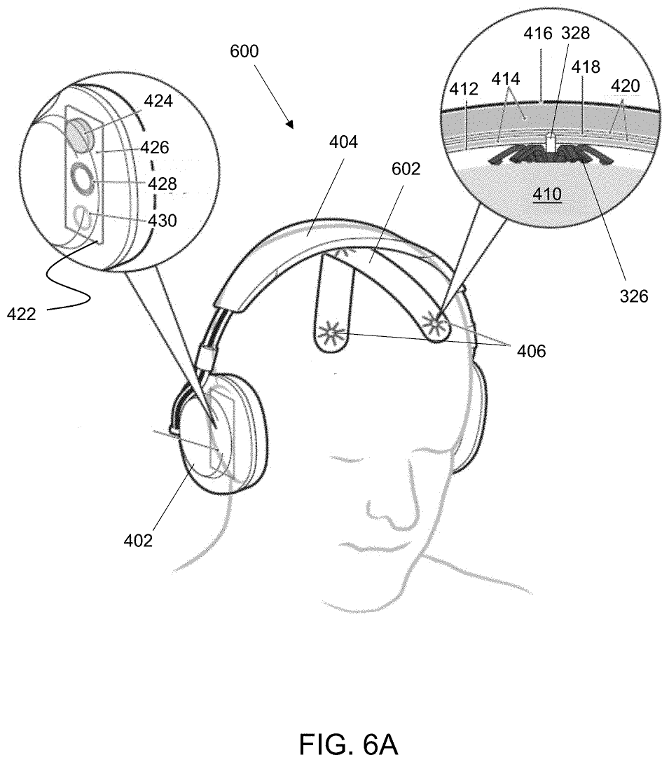

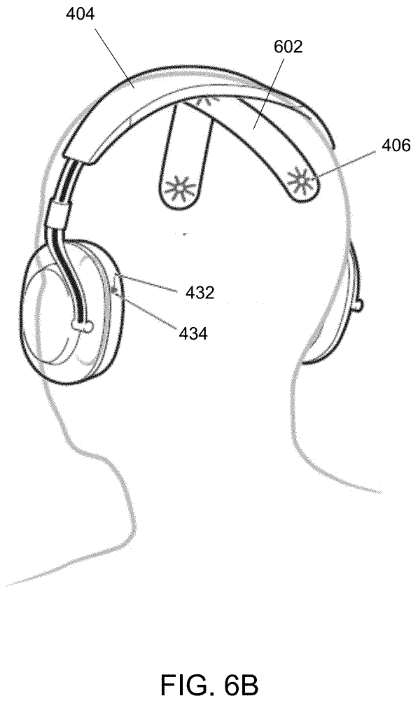

FIGS. 6A and 6B are respectively schematic front and rear diagrams illustrating a wearable unit 600 that can be used in another embodiment of the invention. Features in common with FIGS. 4A and 4B are given the same reference number and are not described again. In this example, the wearable unit 600 comprises a cross-shaped head engagement element 602 in place of the halo shown in FIGS. 4A and 4B. In this example, the head engagement element 602 comprises a pair of elongate strips, each of which is pivotably attached at a middle region thereof to an underside of the head band 404 of the headphones 402. Each strip may be from flexible or deformable material to enable it to conform to the shape of the user's head when worn. The pivotable mounting on the head band enables the strips to be rotated, thereby permitting adjustment of the sensor locations on the user's head.

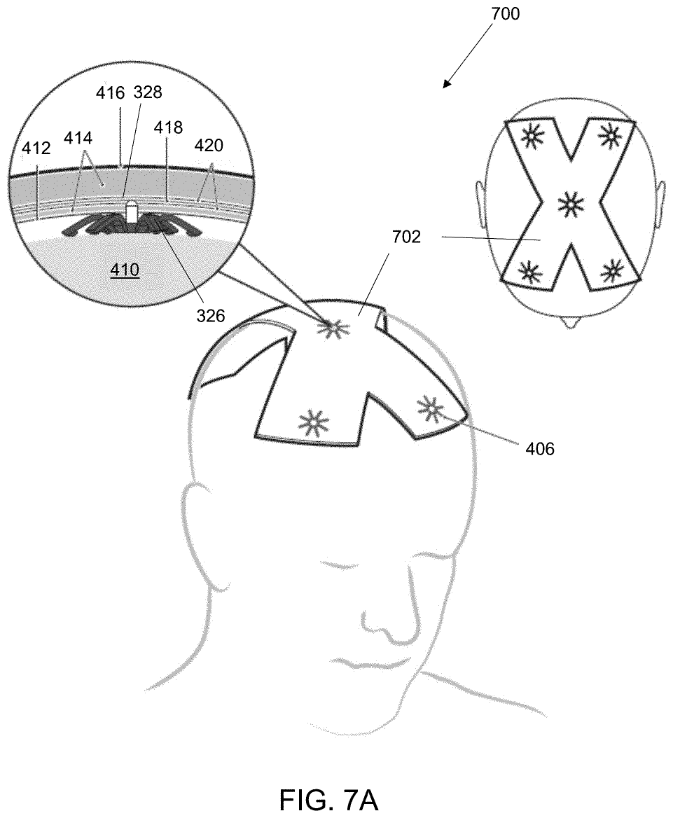

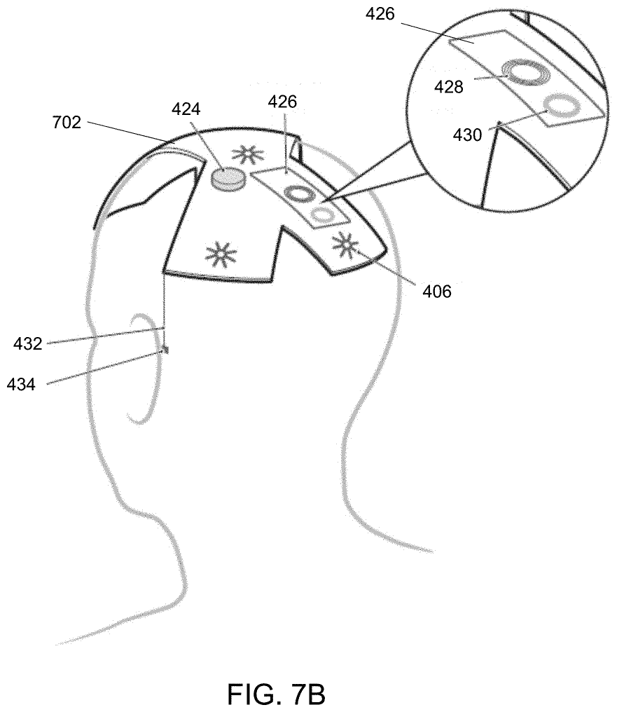

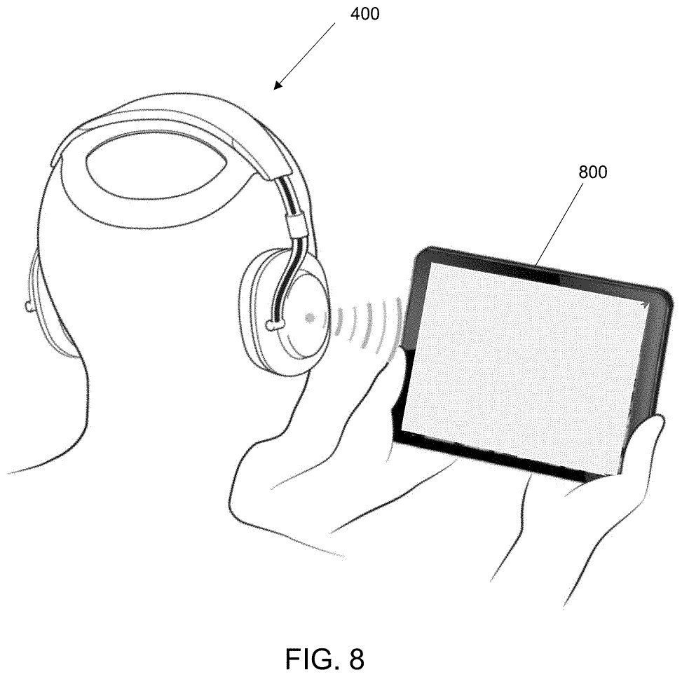

FIGS. 7A and 7B are respectively schematic front and rear diagrams illustrating a wearable unit 700 that can be used in another embodiment of the invention. Features in common with FIGS. 4A and 4B are given the same reference number and are not described again. In this example, the wearable unit 700 need not be used in conjunction with an audio playback device (such as headphones), but rather provide a standalone detection device for reading and wireless communicating an EEG signal. The wearable unit 700 comprises a cross-shaped head engagement element 702 formed from a flexible or deformable material that can conform to the shape of the user's head when worn. The head engagement element 702 may be secured on the user's head in any suitable manner, e.g. using clips or the like. The head engagement element 702 may be worn under conventional headgear.

FIG. 8 is a schematic diagram of a system that is an embodiment of the invention in use. A user wears a wearable unit 400, such as that discussed above with respect to FIGS. 4A and 4B. The wearable unit 400 is in wireless communication with a portable computing device (e.g. a tablet computer) 800 on which the user can consume media content. In one example, the user may watch video content on the portable computing device while the audio content is communicated to and played back through the headphones of the wearable unit 400. The sensors in the wearable unit may detect an EEG signal for the user, and send it to the portable computing device, which may run a user interface application as discussed above to determine mental state information for the user. The mental state information may be used to assist the user in rating consumed content, or to recommend other content that matches the user's mood. In addition, the mental state information gathered while a user is consuming content may be synchronised with that content, and used to create a repository of annotated media content that can be matched to a user's future mental state.

1. A system comprising:
a head-mountable wearable sensor comprising:
a sensor array arranged to detect an electroencephalographic (EEG) signal from a user wearing the wearable sensor;
a filter module arranged to recognise and remove artefact waveforms from the EEG signal to generate a filtered EEG signal; and
a communication unit wirelessly transmitting the filtered EEG signal; and
a processing unit arranged to receive the filtered EEG signal transmitted from the head-mountable wearable sensor,
wherein the processing unit comprises an analyser module arranged to generate, based on the filtered EEG signal, output data that is indicative of mental state information for the user, and
wherein the wearable sensor is incorporated into headgear worn by the user exposed to a real world and/or virtual reality external stimulus, whereby the output data provides real-time mental state information for the user while exposed to the external stimulus.

2. The system according to claim 1, wherein the filter module is arranged to apply a recognition algorithm to the EEG signal to filter out waveforms associated with certain artefacts, and wherein the filter module is adapted to update the recognition algorithm using specific waveform for each type of artefact obtained for the user.

3. The system according to claim 1, wherein the processing unit comprises a correlator module arranged to correlate the mental state information with the external stimulus.

4. The system according to claim 3, wherein the processing unit is arranged to time stamp the mental state information, and arranged to synchronise the time stamped mental state information with data indicative of the external stimulus.

5. The system according to claim 4, wherein the data indicative of the external stimulus comprises a time series of annotatable events that correspond to the external stimulus.

6. The system according to claim 3, wherein the external stimulus comprising exposure to media content, and wherein the correlator module is arranged synchronise the mental state information with the media content.

7. The system according to claim 3, comprising a repository for storing the correlated mental state information.

8. The system according to claim 1 further comprising a portable computing device arranged to execute a user interface application to enable user interaction with the output data.

9. The system according to claim 8, wherein the processing unit is part of the portable computing device.

10. The system according to claim 8, wherein the user interface application is arranged to recommend a rating for the external stimulus based on the output data.

11. The system according to claim 8, wherein the user interface application is arranged to suggest user action based on the output data.

12. The system according to claim 11, wherein the suggested user action comprises any one or more of:
playback of media content,
streaming of media content,
participation in an activity, and
selection or purchase of a retail item or retail service.

13. The system according to claim 8, wherein the user interface application is arranged to compare current output data with historical output data for the user.

14. The system according to claim 1, wherein the analyser module comprises a model configured to map data from the filtered EEG signal onto a mental state vector, wherein the model is adaptive to learn how the user's individual EEG signals map on to emotional state information.

15. The system according to claim 14, wherein the mental state vector comprises components that are each indicative of an intensity value or probability for a respective emotional state or mental process.

16. The system according to claim 14, wherein the data from the filtered EEG signal comprises first data indicative of asymmetry in the Alpha and Beta EEG bands across the left hemispheric bank and second data indicative of asymmetry in the Alpha and Beta EEG bands across the right hemispheric bank.
